(19) United States
(12) Patent Application Publication
(10) Pub. No.: US 2021/0114975 A1
(43) Pub. Date: Apr. 22, 2021

(54) THERAPEUTIC COMPOUND AND METHODS

(71) Applicant: Regents of the University of Minnesota, Minneapolis, MN (US)

(72) Inventor: Venkatram R. Mereddy, Minneapolis, MN (US)

(73) Assignee: Regents of the University of Minnesota, Minneapolis, MN (US)

(21) Appl. No.: 17/076,527

(22) Filed: Oct. 21, 2020

Related U.S. Application Data
(60) Provisional application No. 62/924,032, filed on Oct. 21, 2019.

(51) Int. Cl. C07C 255/23; A61P 35/00

(52) U.S. Cl. CPC C07C 255/23; A61P 35/00

(57) ABSTRACT

The invention provide a compound of formula I:

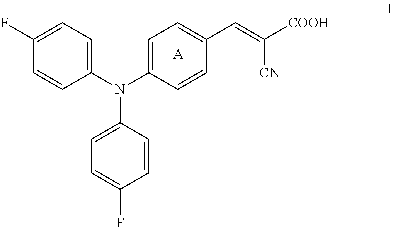

or a salt thereof. The invention also provides pharmaceutical compositions comprising a compound of formula I or a pharmaceutically acceptable salt thereof, processes for preparing a compound of formula I or a pharmaceutically acceptable salt thereof, intermediates useful for preparing a compound of formula I or a pharmaceutically acceptable salt thereof, and therapeutic methods comprising the administration of a compound of formula I or a pharmaceutically acceptable salt thereof. The compounds and salts are useful for inhibiting glycolysis and mitochondrial function and are useful for treating cancer, autoimmune diseases, NASH, CGvHD, and obesity. The compounds and salts are are useful for treating transplant rejection.

### RELATED APPLICATION

This application claims priority to U.S. Provisional Application No. 62/924,032 that was filed on Oct. 21, 2019. The entire content of the application referenced above is hereby incorporated by reference herein.

### BACKGROUND OF THE INVENTION

Metabolic plasticity between glycolysis and mitochondrial OxPhos has become an important biological target for the development of new therapeutics for the treatment of cancer. Most of the known compounds that target mitochondria involve conjugating a cationic lipophilic agent such as triphenyl phosphine to a cytotoxic agent. Although these conjugates exhibit excellent cytotoxicity, they suffer from narrow therapeutic index, making them unsuitable for human use.

International Patent Application Publication Number WO/2013/109972 describes compounds that are reported to be useful for treating or preventing cancer, treating or preventing autoimmune diseases, or that are useful for preventing transplant rejection.

There is a current need for agents that are useful for treating or preventing cancer, autoimmune diseases, NASH, CGvHD, or obesity. In particular, there is a need for agents with improved properties (e.g. a wider therapeutic index).

### SUMMARY OF THE INVENTION

Applicant has identified compounds with excellent potency in inhibiting glycolysis and mitochondrial function in addition to a high therapeutic index.

Accordingly, in one embodiment, the invention provides a compound of the invention which is a compound of formula I:

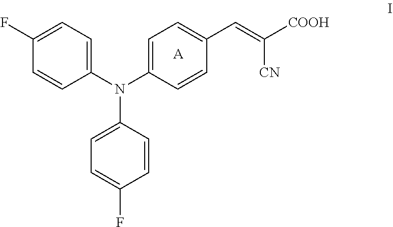

or a salt thereof, wherein ring A is optionally substituted with one or more groups independently selected from the group consisting of hydroxy, halo, cyano, (C₁-C₃)alkyl, (C₁-C₃)alkoxy, (C₁-C₃)alkanoyl, (C₁-C₃)alkanoyloxy, (C₁-C₃)alkoxycarbonyl, (C₃-C₆)cycloalkyl, (C₃-C₆)cycloalkyl(C₁-C₃)alkyl.

The invention also provides a pharmaceutical composition comprising a compound of formula I or a pharmaceutically acceptable salt thereof, and a pharmaceutically acceptable diluent or carrier.

The invention also provides a method for inhibiting cancer (e.g. lung, breast, brain, prostate, pancreatic, colorectal, ovarian, head and neck) cell growth comprising contacting the cancer cell in vitro or in vivo with a compound of formula I or a salt thereof.

The invention also provides a method for treating cancer (e.g. lung, breast, brain, prostate, pancreatic, colorectal, ovarian, head and neck cancer) in a mammal (e.g. a human) comprising administering to the mammal an effective amount of compound of formula I or a pharmaceutically acceptable salt thereof.

The invention also provides a method for treating an autoimmune disease (e.g. rheumatoid arthritis) in a mammal (e.g. a human) comprising administering to the mammal a compound of formula I or a pharmaceutically acceptable salt thereof.

The invention also provides a method for preventing transplant rejection (e.g. heart, kidney, eye, liver, lung, pancreas, intestine, and thymus transplant rejection) or tissue graft rejection (e.g. bone, tendon, cornea, skin, heart valve and vein) in a mammal (e.g. a human) comprising administering to the mammal a compound of formula I, or a pharmaceutically acceptable salt thereof.

The invention also provides a method for diagnosing cancer (e.g. lung, breast, brain, prostate, pancreatic, colorectal, ovarian, head and neck cancer) or for diagnosing an autoimmune disease (e.g. rheumatoid arthritis) in a mammal comprising administering to the mammal (e.g. a human) a compound of formula I, or a pharmaceutically acceptable salt thereof and measuring or imaging the fluorescence of the compound of formula I or the salt, wherein the fluorescence correlates with cancer or an autoimmune disease.

The invention also provides a method for imaging cancerous cells (e.g. brain, breast, head, neck, lung or colon cancer cells) or for imaging cells involved in an autoimmune response (e.g. rheumatoid arthritis) or for imaging cells involved in transplant rejection (e.g. heart, kidney, eye, liver, lung, pancreas, intestine, and thymus transplant rejection) or tissue graft rejection (e.g. bone, tendon, cornea, skin, heart valve and vein) comprising contacting the cells in vivo or in vitro with a compound of formula I or a salt thereof and imaging the fluoroscence of the compound of formula I or the salt while in contact with the cell.

The invention also provides a method for altering brain function (e.g. long-term memory formation) in a mammal (e.g. a human) comprising administering to the mammal a compound of formula I or a pharmaceutically acceptable salt thereof.

The invention also provides a method for treating NAFLD in a mammal comprising administering to the mammal a compound of formula I or a pharmaceutically acceptable salt thereof.

The invention also provides a method for treating CGvHD in a mammal comprising administering to the mammal a compound of formula I or a pharmaceutically acceptable salt thereof.

The invention also provides a method for treating obesity in a mammal comprising administering to the mammal a compound of formula I or a pharmaceutically acceptable salt thereof.

The invention also provides a compound of formula I or a salt thereof for use in diagnosing cancer (e.g. lung, breast, brain, prostate, pancreatic, colorectal, ovarian, head and neck) or for diagnosing a autoimmune disease (e.g. rheumatoid arthritis).

The invention also provides a compound of formula I or a salt thereof for use in imaging cancerous cells (e.g. brain, breast, head, neck, lung or colon cancer cells) or for imaging cells involved in an autoimmune response (e.g. rheumatoid arthritis) or for imaging cells involved in transplant rejection (e.g. heart, kidney, eye, liver, lung, pancreas, intestine, and thymus transplant rejection) or tissue graft rejection (e.g. bone, tendon, cornea, skin, heart valve and vein).

The invention also provides processes and intermediates disclosed herein that are useful for preparing a compound of formula I or a salt thereof.

### BRIEF DESCRIPTION OF THE FIGURES

FIGS. 1A-1B. Mitochondrial stress test using compound 1 (FMD-1) in (1A) MCT4 expressing MDA-MB-231 and (1B) MCT1 expressing WiDr cell lines.

FIGS. 2A-2D. Mitochondrial stress test of compound 1 (FMD-1) in MDA-MB-231 cell line. The graphs represent mitochondrial parameters (2A) maximal respiration (2B) proton leak (2C) ATP production, and (2D) spare respiratory capacity. *, P<0.05, **, P<0.01, ***, P<0.001, ****, P<0.0001.

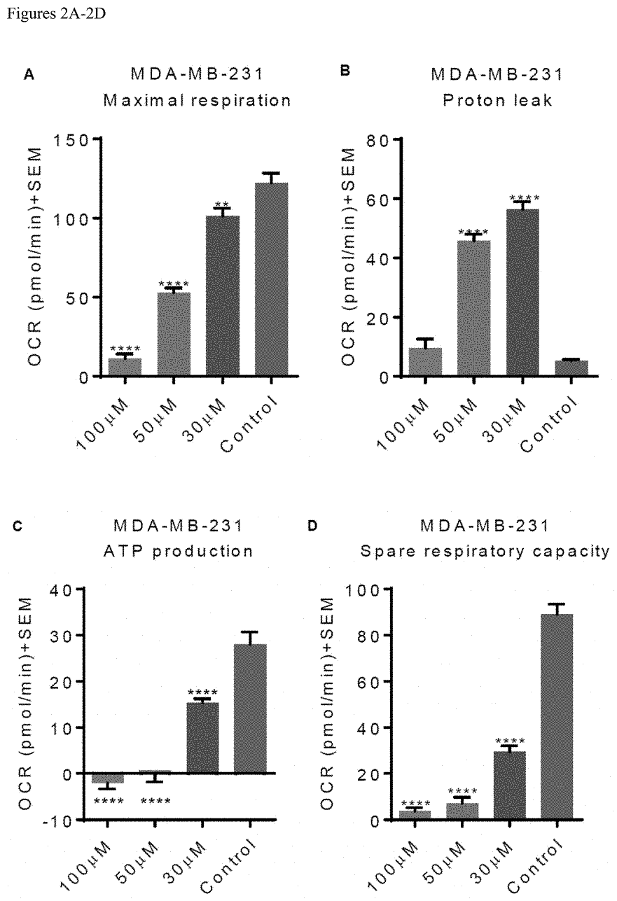

FIGS. 3A-3D. Mitochondrial stress test of compound 1 (FMD-1) in WiDr cell line. The graphs represent mitochondrial parameters (3A) maximal respiration (3B) proton leak (3C) ATP production, and (3D) spare respiratory capacity. *, P<0.05, **, P<0.01, ***, P<0.001, ****, P<0.0001.

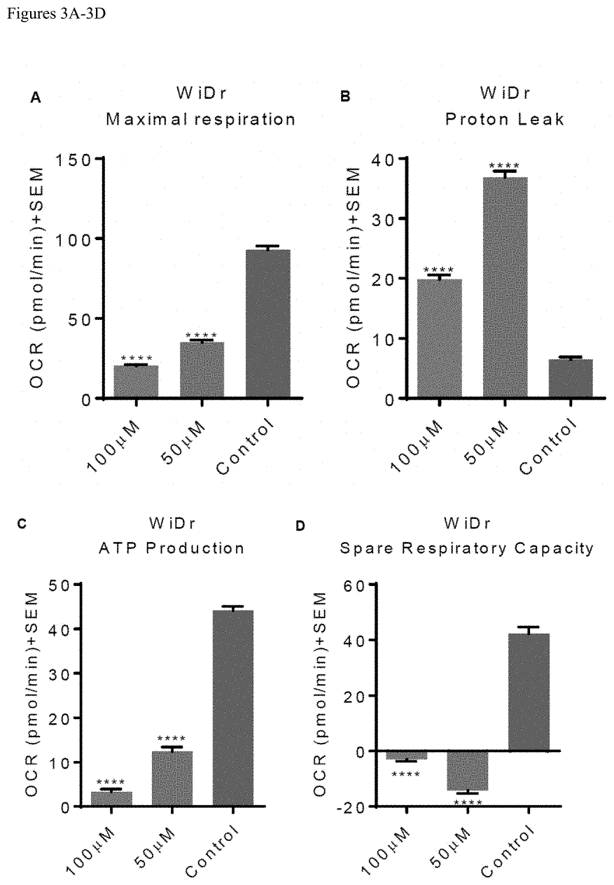

FIG. 4. Glycolysis stress test using compound 1 (FMD-1) in MCT4 expressing MDA-MB-231 cell line.

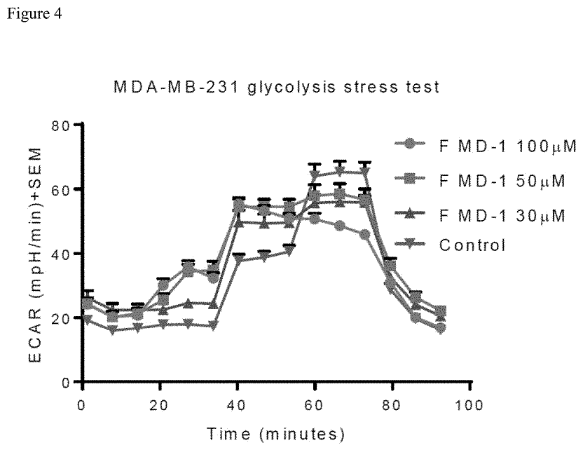

FIGS. 5A-5C. Glycolysis stress test of compound compound 1 (FMD-1) in MDA-MB-231 cell line. The graphs represent glycolytic parameters (5A) glycolysis (5B) glycolytic capacity and (5C) glycolytic reserve. *, P<0.05, **, P<0.01, ***, P<0.001, ****, P<0.0001.

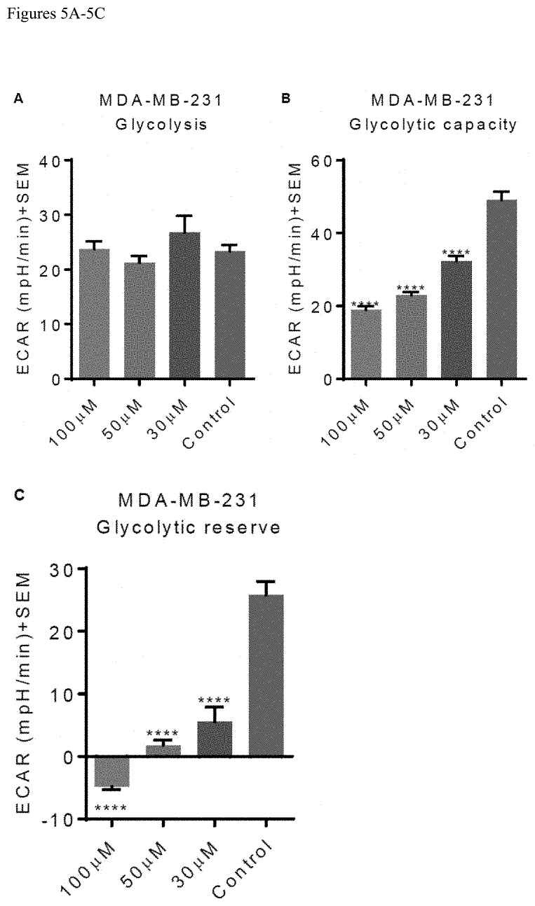

FIGS. 6A-6B. Candidate compounds inhibit pyruvate-driven respiration in (6A) highly oxygen consuming and MPC1/2 expressing 4T1 cells. (6B) Microscopy experiments illustrate that rPFO (1 nM) effectively permeabilized 4T1 cells as indicated by propidium iodide uptake without altering MitoTracker™ Red fluorescence (See Example 4).

FIG. 7. Compound 2 inhibits pyruvate-driven respiration in permeabilized cells in a dose dependent fashion (Example 4).

FIG. 8. Dose-response curve for Compound 2 (Example 4).

### DETAILED DESCRIPTION

The following definitions are used, unless otherwise described.

The term “alkyl”, by itself or as part of another substituent, means, unless otherwise stated, a straight or branched chain hydrocarbon radical, having the number of carbon atoms designated (i.e., (C₁-C₆) means one to six carbons). Examples include (C₁-C₆)alkyl, (C₂-C₆)alkyl, C₂-C₆)alkyl, (C₃-C₆)alkyl and (C₄-C₆)alkyl. Examples of alkyl groups include methyl, ethyl, n-propyl, iso-propyl, n-butyl, t-butyl, iso-butyl, sec-butyl, n-pentyl, and n-hexyl.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 6 carbon atoms (i.e., (C₃-C₆)carbocycle).

The term “alkanoyl” as used herein refers to a group (alkyl)-C(═O)—, wherein the term alkyl has the meaning defined herein.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

The term “NAFLD” as used herein refers to nonalcoholic fatty liver disease (NAFLD), a condition in which fat builds up in the liver. The term includes Nonalcoholic steatohepatitis (NASH).

The term “CGvHD” as used herein includes graft vs host disease, when donor bone marrow or stem cells attack a bone marrow or stem cell transplant recipient.

It will be appreciated by those skilled in the art some compounds may exhibit polymorphism. It is to be understood that the present invention encompasses any polymorphic or stereoisomeric form, or mixtures thereof, of a compound of the invention, which possess the useful properties described herein

A salt of a compound of formula I can be useful as an intermediate for isolating or purifying the compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compound of formula I or a pharmaceutically acceptable salt thereof can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compound or a pharmaceutically acceptable salt thereof may be systemically administered, e.g., orally, in combination with a pharmaceutically acceptable vehicle such as an inert diluent or an assimilable edible carrier. It may be enclosed in hard or soft shell gelatin capsules, may be compressed into tablets, or may be incorporated directly with the food of the patient's diet. For oral therapeutic administration, the active compound or a pharmaceutically acceptable salt thereof may be combined with one or more excipients and used in the form of ingestible tablets, buccal tablets, troches, capsules, elixirs, suspensions, syrups, wafers, and the like. Such compositions and preparations should contain at least 0.1% of active compound or a salt thereof. The percentage of the compositions and preparations may, of course, be varied and may conveniently be between about 2 to about 60% of the weight of a given unit dosage form. The amount of active compound or a salt thereof in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound or a salt thereof, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound or a pharmaceutically acceptable salt thereof may be incorporated into sustained-release preparations and devices.

The active compound or a pharmaceutically acceptable salt thereof may also be administered intravenously or intraperitoneally by infusion or injection. Solutions of the active compound or a pharmaceutically acceptable salt thereof can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound or a pharmaceutically acceptable salt thereof in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compound or a pharmaceutically acceptable salt thereof may be applied in pure form, i.e., when they are liquids. However, it will generally be desirable to administer the active ingredient to the skin as a composition or formulation, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compound or salt can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compound of formula I or a pharmaceutically acceptable salt thereof to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of formula I or a pharmaceutically acceptable salt thereof can be determined by comparing its in vitro activity, and in vivo activity in animal models. Methods for the extrapolation of effective dosages in mice, and other animals, to humans are known to the art; for example, see U.S. Pat. No. 4,938,949.

The amount of the compound or a pharmaceutically acceptable salt thereof required for use in treatment will vary not only with the particular salt selected but also with the route of administration, the nature of the condition being treated and the age and condition of the patient and will be ultimately at the discretion of the attendant physician or clinician.

The compound or a pharmaceutically acceptable salt thereof is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention or a pharmaceutically acceptable salt thereof formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Aggressive glycolysis is the hallmark of all advanced stage tumors. Accordingly, the compound, salts, and compositions described herein may be particularly useful to treat cancers that express MCT's (e.g. lung, breast, brain, prostate, pancreatic, colorectal, ovarian, head and neck).

In addition, the compound, salts, and compositions described herein may be useful to prevent organ (e.g. heart, kidney, eye, liver, lung, pancreas, intestine, and thymus) transplant rejection as well as tissues graft (e.g. bone, tendon, cornea, skin, heart valves, and veins) rejection. The compound, salts, and compositions described herein may also be useful for treating autoimmune diseases (e.g. rheumatoid arthritis).

The compound of the invention or a pharmaceutically acceptable salt thereof can also be administered in combination with other therapeutic agents, for example, other agents that are useful for the treatment of cancer (e.g. lung, breast, brain, prostate, pancreatic, colorectal, ovarian, head and neck cancer) or autoimmune diseases (e.g. rheumatoid arthritis) or agents that are useful for preventing transplant rejection such as organ transplant (e.g. heart, kidney, eye, liver, lung, pancreas, intestine, and thymus) rejection and or tissue graft (e.g. bone, tendon, cornea, skin, heart valve, and vein) rejection. Accordingly, in one embodiment the invention also provides a composition comprising a compound of formula I or a pharmaceutically acceptable salt thereof, at least one other therapeutic agent, and a pharmaceutically acceptable diluent or carrier. The invention also provides a kit comprising a compound of formula I or a pharmaceutically acceptable salt thereof, at least one other therapeutic agent, packaging material, and instructions for administering the compound of formula I or the pharmaceutically acceptable salt thereof and the other therapeutic agent or agents to an animal to treat cancer or an autoimmune disease or to prevent transplant rejection.

In one embodiment, the compound or salt is selected from the group consisting of:

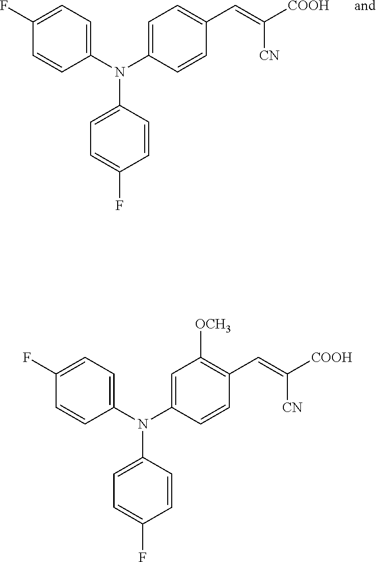 [SMILES: N#C/C(=C\c1ccc(N(c2ccc(F)cc2)c2ccc(F)cc2)cc1)C(=O)O ; "and" ; COc1cc(N(c2ccc(F)cc2)c2ccc(F)cc2)ccc1/C=C(\C#N)C(=O)O]

and salts thereof.

In one embodiment, the compound or salt is:

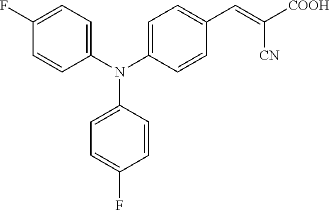 [SMILES: N#C/C(=C\c1ccc(N(c2ccc(F)cc2)c2ccc(F)cc2)cc1)C(=O)O]

or a pharmaceutically acceptable salt thereof.

In one embodiment, the compound or salt is:

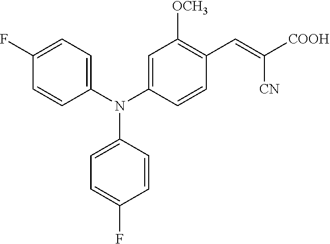 [SMILES: COc1cc(N(c2ccc(F)cc2)c2ccc(F)cc2)ccc1/C=C(\C#N)C(=O)O]

or a pharmaceutically acceptable salt thereof.

In one embodiment, the compound or salt is a compound of formula (Ia):

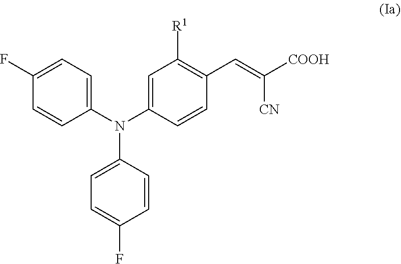

or a salt thereof, wherein R¹ is selected from the group consisting of hydroxy, halo, cyano, (C₁-C₃)alkyl, (C₁-C₃)alkoxy, (C₁-C₃)alkanoyl, (C₁-C₃)alkanoyloxy, (C₁-C₃)alkoxycarbonyl, (C₃-C₆)cycloalkyl, (C₃-C₆)cycloalkyl(C₁-C₃)alkyl. In one embodiment, R¹ is (C₁-C₃)alkoxy.

The invention will now be illustrated by the following non-limiting Examples.

### EXAMPLES

### Example 1. Synthesis of (E)-3-(4-(bis(4-fluorophenyl)amino)phenyl)-2-cyanoacrylic Acid (1)

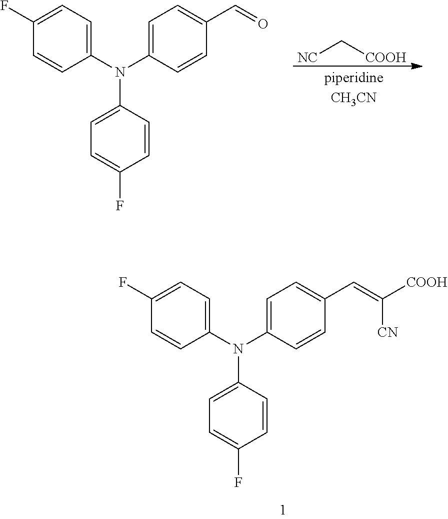 [SMILES: O=Cc1ccc(N(c2ccc(F)cc2)c2ccc(F)cc2)cc1 ; N#CCC(=O)O ; N#C/C(=C\c1ccc(N(c2ccc(F)cc2)c2ccc(F)cc2)cc1)C(=O)O]

To a stirred solution of 4-(bis(4-fluorophenyl)amino)benzaldehyde (3 mmol) in acetonitrile (15 mL), piperidine (3.2 mmol) and cyanoacetic acid (3.2 mmol) were added. The reaction mixture was refluxed at 80° C. for 6 hours. Upon completion, reaction mixture was quenched with ice cold 3N HCL and extracted with ethyl acetate and the crude compound was purified via column chromatograph (7:3 ethyl acetate/hexane) to provide the product. ¹H NMR (500 MHz, DMSO-d₆): δ 7.78-7.73 (m, 3H), 7.23-7.14 (m, 8H), 6.82 (d, J=8.5 Hz, 2H). ¹³C NMR (125 MHz, DMSO-d₆): δ 163.64, 160.56, 158.63, 149.93, 146.58, 142.81, 142.79, 131.27, 128.29, 128.22, 126.19, 120.52, 119.44, 117.25, 117.06, 111.30, 79.72.

### Example 2. Synthesis of (E)-3-(4-(bis(4-fluorophenyl)amino)-2-methoxyphenyl)-2-cyanoacrylic Acid (2, 2FCAA)

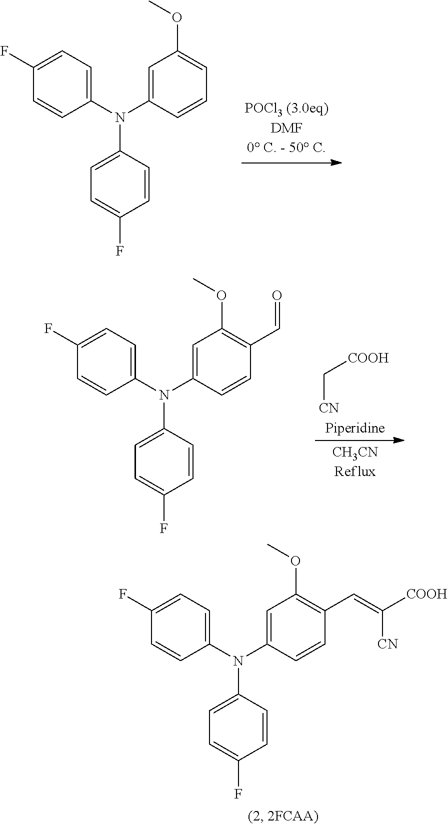 [SMILES: COc1cccc(N(c2ccc(F)cc2)c2ccc(F)cc2)c1 ; COc1cc(N(c2ccc(F)cc2)c2ccc(F)cc2)ccc1C=O ; N#CCC(=O)O ; COc1cc(N(c2ccc(F)cc2)c2ccc(F)cc2)ccc1/C=C(\C#N)C(=O)O]

### a. Synthesis of 4-(bis(4-fluorophenyl)amino)-2-methoxybenzaldehyde

N,N-bis(4-fluorophenyl)-3-methoxyaniline (2 mmol) was dissolved in dimethylformamide (25 mL) and cooled on ice. To the reaction, POCl₃ (6 mmol) was added dropwise. The reaction was taken off ice and allowed to come to room temperature for 1 hour. Upon total consumption of the phenol, the reaction was poured over 100 mL of an ice-cold sodium carbonate (6.5 mmol) and extracted with ethyl acetate (50 mL×3). The organic phase was dried with magnesium sulfate and concentrated under vacuum to yield a dark orange crude oil.

### b. Synthesis of (E)-3-(4-(bis(4-fluorophenyl)amino)-2-methoxyphenyl)-2-cyanoacrylic Acid

The aldehyde (0.6 mmol) was dissolved in acetonitrile followed by addition of cyanoacetic acid (2 mmol). The reaction was cooled on ice followed by the addition of piperidine (0.9 mmol). The reaction was removed from ice and refluxed for 12 hours. Reaction progress was monitored via TLC (40% EtOAc/Hexanes). Upon consumption of the aldehyde mixture was stirred in 150 mL of 2 M HCl for 20 minutes. Product was extracted with ethyl acetate (50 mL×3), dried with anhydrous magnesium sulfate and concentrated under vacuum to give an orange solid. (2FCCA, 67% yield). ¹H NMR (400 MHz, DIMETHYL SULFOXIDE-d6): δ 8.44 (s, 1H), 8.12 (d, J=8.96 Hz, 1H), 7.36-7.26 (m, 8H), 6.37 (dd, J=1.72, 8.92 Hz, 1H), 6.30 (s, 1H), 3.66 (s, 3H). ¹³C NMR (100 MHz, DIMETHYL SULFOXIDE-d6): δ 164.94, 161.07, 160.40 (d, J_{CF}=242.36 Hz), 154.61, 146.93, 141.62 (d, J_{CF}=2.63 Hz), 130.18, 129.52 (d, J_{CF}=8.58 Hz), 117.82, 117.41 (d, J_{CF}=22.66 Hz), 112.07, 110.56, 99.74, 96.82, 56.15. HRMS (ESI) m/z: calc'd for C₂₃H₁₆F₂N₂O₄ [M+H⁺]: 407.1202 found: 407.1229.

### Example 3. Mitochondrial and Glycolysis Stress Tests in MDA-MB-231 and WiDr Cell Line

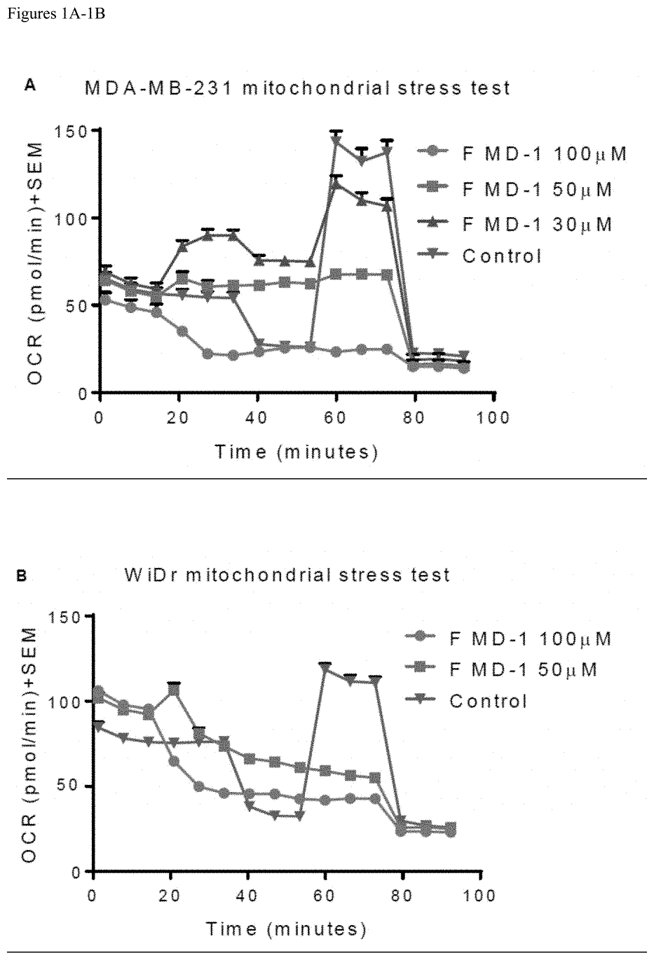

The metabolic profile of compound 1 was evaluated via mitochondrial stress test (MST) and glycolytic stress test (GST) on MCT4 expressing MDA-MB-231 and MCT1 expressing WiDr cell lines using Seahorse XFe96® analyzer (FIGS. 1A and 1B). 20,000 cells/well were seeded in 96-well microplates. MST was performed according to the manufacturer's protocol. The mitochondrial parameters: maximal respiration, ATP production, proton leak and spare respiratory capacity were calculated using wave software. DMSO was used as the negative control. A concentration study was performed in MDA-MB-231 cell line using compound 1 at 100, 50 and 30 μM concentrations. At all three concentrations, maximal respiration, ATP production and spare respiratory capacity were significantly decreased compared to the control and the amount of inhibition is higher at low concentrations in both MDA-MB-231 and WiDr cell lines. GST was also performed according to the manufacturer's protocol. The glycolytic parameters: glycolysis, glycolytic capacity, and glycolytic reserve were calculated using wave software, with DMSO as a negative control. In this case also, a concentration study was carried out at 100, 50 and 30 μM in MDA-MB-231 cell line. Glycolytic capacity and glycolytic reserve parameters were significantly decreased compared to the control, and the amount of inhibition is high at higher concentrations of FMD-1.

### Example 4. Inhibition Properties of Pyruvate-Driven Respiration in 4T1 Cells

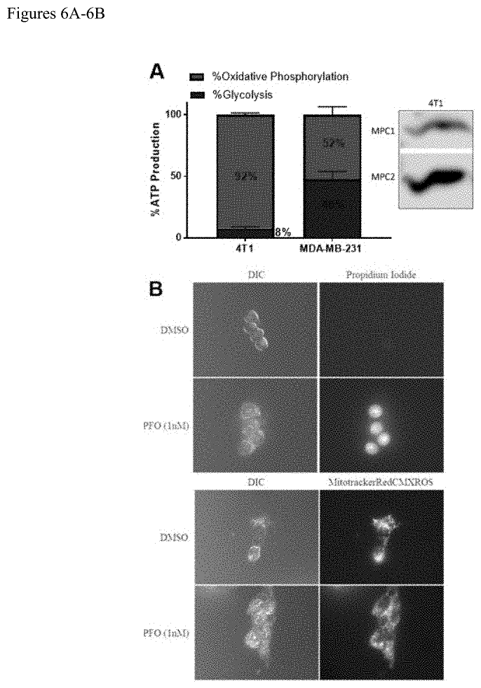

The ability of Compound 2 to inhibit pyruvate-driven respiration in a 4T1 breast cancer cell line was evaluated. This cell line was found to exhibit a substantially high basal level of oxidative phosphorylation when compared to an established glycolytic cancer cell line, MDA-MB-231 (FIG. 6A). Further, this cell line was found to express both MPC1 and MPC2 at readily detectable levels, suitable for screening drug candidates with potential MPC inhibitory capacity (FIG. 6A). To evaluate the ability of Compound 2 to inhibit pyruvate-driven respiration (i.e., MPC inhibition), a slightly modified Seahorse XFe96-based respiratory experiment in permeabilized cells that had been utilized elsewhere was employed. In permeabilized cells, small polar metabolites, including pyruvate, can be delivered directly to mitochondria independent of plasma membrane transport and hence, the kinetics of mitochondrial pyruvate-driven respiration can be measured directly. Recombinant perfringolyyin O (rPFO) is a cytolysin excreted by Clostridium perfringens that potently and acutely permeabilizes the plasma membrane of eukaryotic cells without impacting organelle membrane integrity. To test the ability of rPFO to permeabilize the plasma membrane in the model system, epifluorescence microscopy experiments were employed. Here, 4T1 cells were seeded in MatTek glass-bottom dishes and incubated for 48 hours to allow the cells to adhere. Cells were then exposed to rPFO (1 nM) and incubated for 30 minutes in growth media, a relevant time point in Seahorse experiments. To validate membrane permeability, propidium iodide uptake was monitored. Media was then aspirated and replaced with a mannitol/sucrose buffer (MAS buffer; 70 mM sucrose, 220 mM mannitol, 10 mM potassium phosphate monobasic, 5 mM magnesium chloride, 2 mM HEPES, and 1 mM EGTA) containing both propidium iodide (PI) and MitoTracker™ Red CMXROS (MTR), a probe that binds and accumulates to the mitochondria as a function of membrane potential. PI enabled observation of membrane permeability as intact membranes do not allow PI uptake. MTR was utilized in these experiments to assess the effects of rPFO on mitochondrial viability, as damaged mitochondria will exhibit a dim/diffuse fluorescence. These experiments revealed that rPFO potently (1 nM) and acutely (30 minutes) permeabilized the plasma membranes as indicated by PI uptake in rPFO-treated cells (FIG. 6B). Further, mitochondria remained viable with comparable MTR fluorescent intensity in rPFO cells when compared to controls (FIG. 6B).

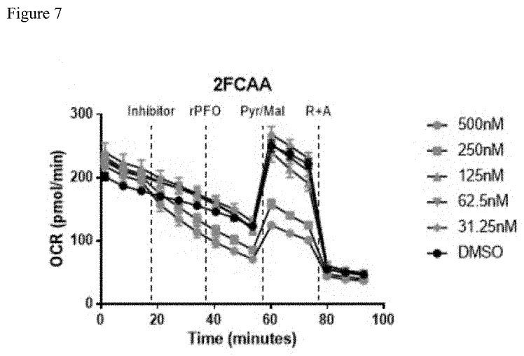

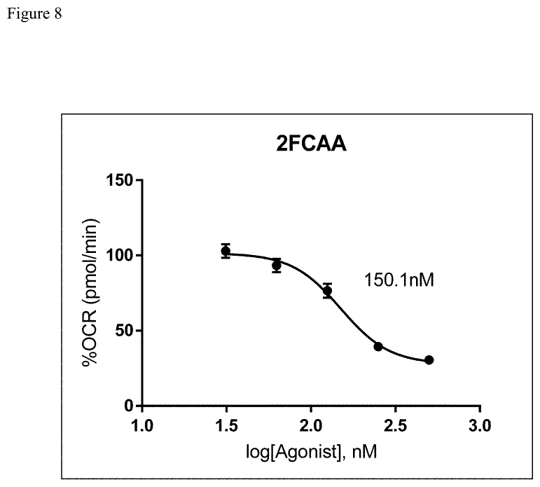

Compound 2 inhibited pyruvate-driven respiration in permeabilized cells in a dose dependent fashion (FIG. 7) enabling a dose-response curve (FIG. 8) to be generated and an IC₅₀ value to be calculated. All data are representative of at least three independent experiments. The IC₅₀ value in FIG. 8 is the average±SEM of three independent experiments (n=3 biological replicates, n=15 technical replicates per dose).

After validating the ability of rPFO to permeabilize 4T1 cells without damaging mitochondria, the ability of Compound 2 to inhibit pyruvate-driven respiration in these cells was evaluated. 4T1 cells were seeded in Seahorse XFe96 plates and were incubated to confluence (˜18-24 hours) in growth media (+serum). Growth media was then aspirated and cells were washed with MAS buffer to remove serum and endogenous metabolic substrates. Serum- and substrate-starved cells were then incubated for equilibration in MAS buffer in a non-CO₂ incubator. Inhibitors, rPFO, and substrate milieus were prepared in MAS buffer to be injected in succession to allow for real-time observation of the effects of compound-treated cultures on oxygen consumption rates (OCR) when compared to vehicle (DMSO)-treated cells. Equilibrated intact cells were allowed to establish basal respiratory rates, followed by the injection of Compound 2, at varying concentrations. Acute decreases in OCR were observed (FIG. 7). Compound exposure was performed on intact cells to avoid influence of rPFO on acute cellular targets. Cells were then exposed to rPFO, followed by the FCCP-stimulated pyruvate substrate milieu which consisted of pyruvate (Pyr), malate (Mal), and dichloroacetate (DCA) to fuel uninhibited pyruvate respiratory flux and maximal respiration. Malate and DCA were included to allow for continuous TCA cycle function without acetyl CoA feedback-mediated inhibition (malate) or pyruvate dehydrogenase regulated inhibition (DCA) of pyruvate uptake. A dose-dependent inhibition of pyruvate-driven respiration in cells treated with the candidate compound was observed. A logarithmic dose-response curve was generated (FIG. 8) and an IC₅₀ value was calculated.

### Example 5

The following illustrate representative pharmaceutical dosage forms, containing the compound of formula I or a pharmaceutically acceptable salt thereof (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q. s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula I: 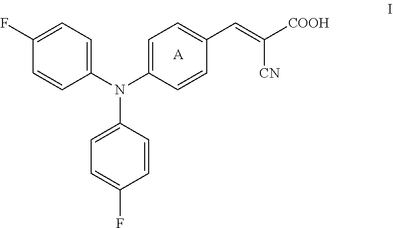
or a salt thereof, wherein ring A is optionally substituted with one or more groups independently selected from the group consisting of hydroxy, halo, cyano, (C₁-C₃)alkyl, (C₁-C₃)alkoxy, (C₁-C₃)alkanoyl, (C₁-C₃)alkanoyloxy, (C₁-C₃)alkoxycarbonyl, (C₃-C₆)cycloalkyl, (C₃-C₆)cycloalkyl(C₁-C₃)alkyl.

2. The compound or salt of claim 1, which is selected from the group consisting of: 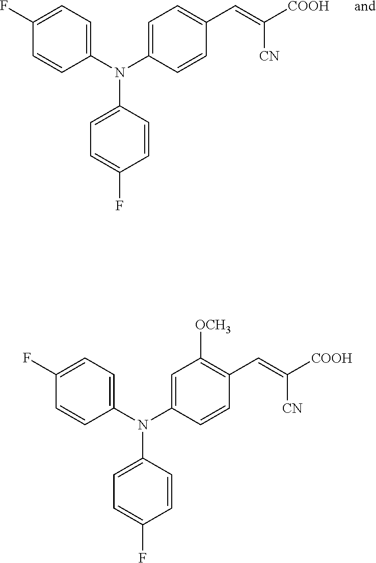 [SMILES: N#C/C(=C\c1ccc(N(c2ccc(F)cc2)c2ccc(F)cc2)cc1)C(=O)O ; "and" ; COc1cc(N(c2ccc(F)cc2)c2ccc(F)cc2)ccc1/C=C(\C#N)C(=O)O]
and salts thereof.

3. The compound or salt of claim 1, which is: 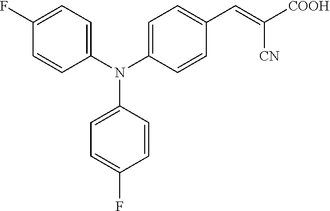 [SMILES: N#C/C(=C\c1ccc(N(c2ccc(F)cc2)c2ccc(F)cc2)cc1)C(=O)O]
or a pharmaceutically acceptable salt thereof.

4. The compound or salt of claim 1, which is: 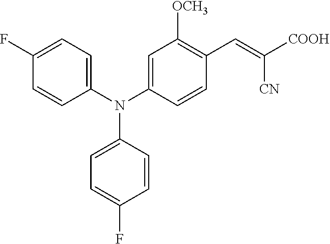 [SMILES: COc1cc(N(c2ccc(F)cc2)c2ccc(F)cc2)ccc1/C=C(\C#N)C(=O)O]
or a pharmaceutically acceptable salt thereof.

5. The compound or salt of claim 1, which is a compound of formula (Ia): 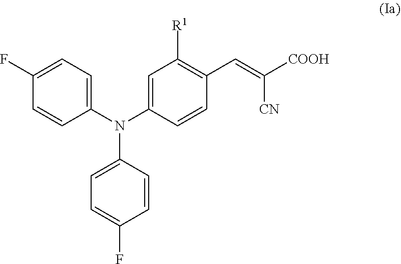
or a salt thereof, wherein R¹ is selected from the group consisting of hydroxy, halo, cyano, (C₁-C₃)alkyl, (C₁-C₃)alkoxy, (C₁-C₃)alkanoyl, (C₁-C₃)alkanoyloxy, (C₁-C₃)alkoxycarbonyl, (C₃-C₆)cycloalkyl, (C₃-C₆)cycloalkyl(C₁-C₃)alkyl.

6. The compound or salt of claim 5, wherein R¹ is (C₁-C₃)alkoxy.

7. A composition comprising a compound of formula I as described in claim 1 or a pharmaceutically acceptable salt thereof, and a pharmaceutically acceptable diluent or carrier.

8. A method for inhibiting cancer cell growth comprising contacting the cancer cell in vitro or in vivo with a compound of formula I as described in claim 1 or a salt thereof.

9. A method for treating cancer in a mammal comprising administering to the mammal a compound of formula I as described in claim 1 or a pharmaceutically acceptable salt thereof.

10. The method of claim 7, wherein the cancer is lung, breast, brain, prostate, pancreatic, colorectal, ovarian, head or neck cancer.

11. A method for treating an autoimmune disease in a mammal comprising administering to the mammal a compound of formula I as described in claim 1 or a pharmaceutically acceptable salt thereof.

12. The method of claim 9, wherein the autoimmune disease is rheumatoid arthritis.

13. A method for preventing transplant rejection or tissue graft rejection in a mammal comprising administering to the mammal a compound of formula I as described in claim 1 or a pharmaceutically acceptable salt thereof.

14. The method of claim 10, wherein the transplant rejection is heart, kidney, eye, liver, lung, pancreas, intestine or thymus transplant rejection and the tissue graft rejection is bone, tendon, cornea, skin, heart valve or vein tissue graft rejection.

15. A method for treating NASH in a mammal comprising administering to the mammal a compound of formula I as described in claim 1 or a pharmaceutically acceptable salt thereof.

16. A method for treating CGvHD in a mammal comprising administering to the mammal a compound of formula I as described in claim 1 or a pharmaceutically acceptable salt thereof.

17. A method for treating obesity in a mammal comprising administering to the mammal a compound of formula I as described in claim 1 or a pharmaceutically acceptable salt thereof.
